SEQUENCE LISTING (1 sequence)

SEQ ID NO: 1 - protein, 35 aa, Artificial sequence
  Phor21-beta-CG(ala) Peptide
KFAKFAKKFAKFAKKFAKFAKSYAVASAQAALAAR

(19) United States
(12) Patent Application Publication
(10) Pub. No.: US 2009/0061006 A1
(43) Pub. Date: Mar. 5, 2009

(54) Layered Nanoparticles for Sustained Release of Small Molecules

(76) Inventors: Carola Leuschner, Baton Rouge, LA (US); Yuri M. Lvov, Ruston, LA (US); Challa S.S.R. Kumar, Baton Rouge, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 12/294,359

(22) PCT Filed: Mar. 28, 2007
(86) PCT No.: PCT/US07/65352

§ 371 Date: Nov. 7, 2008

Related U.S. Application Data
(60) Provisional application No. 60/787,849, filed on Mar. 31, 2006.

(51) Int. Cl. A61K 9/14; A61K 38/17; A61K 38/10

(52) U.S. Cl. 424/491; 424/497; 514/15; 514/14; 514/13; 514/12

(57) ABSTRACT

Nanoparticle compositions and methods are disclosed for the sustained release of small molecules, such as pharmaceutical compounds in vivo, for example ligand-lytic peptide conjugates. The construction of the nanoparticles helps to prevent self-aggregation of the molecules, and the consequent loss of effectiveness. The system employs layer-by-layer self-assembly of biocompatible polyelectrolyte layers, and layers of charged small molecules such as drug molecules, to form a multilayer nanoparticle in which the drug or other small molecule itself acts as one of the alternating charged layers in the multilayer assembly. The small molecules can then be released over time in a sustained manner. The LbL nano-assemblies can specifically target cancers, metastases, or other diseased tissues, while minimizing side effects.

(In countries other than the United States:) The benefit of the 31 Mar. 2006 filing date of U.S. provisional patent application 60/787,849 is claimed under applicable treaties and conventions. (In the United States:) The benefit of the 31 Mar. 2006 filing date of U.S. provisional patent application 60/787,849 is claimed under 35 U.S.C. § 119(e).

The development of this invention was partially funded by the United States Government under Grant BES-0210298 awarded by the National Science Foundation. The Government has certain rights in this invention.

### TECHNICAL FIELD

This invention pertains to layered nanoparticles for the sustained release of small molecules, such as pharmaceutical compounds.

### BACKGROUND ART

There is an unfilled need for improved treatments for cancers and metastases. There is also an unfilled need for improved systems for the sustained release of small molecules, such as drug molecules to treat diseased tissues other than cancers and metastases. Current treatments with small molecule drugs often require multiple bolus injections, because the drug molecules can often self-aggregate and lose inactivity at higher concentrations, or they can be insoluble at higher concentrations, or they otherwise lose activity rapidly under physiological conditions.

Nanoparticles are the subject of current research in biomedical and biotechnological applications. Nanometer-sized particles can offer distinct advantages for drug delivery. Nanoparticles can penetrate deep into tissues through fine capillaries, and can even penetrate into cells. Common materials used in fabricating nanoparticles include iron oxide, gold, silica, and various polymers. The surfaces of the nanoparticles may be modified. For example, the surfaces of silica particles have been modified with avidin, sulfide, amine, and carboxylate groups. These moieties can not only facilitate bioconjugation, but they can also introduce surface charges that may be used in LbL nanoassembly. Silica nanoparticles have been used in biomarkers for cell imaging, in biosensors, in DNA detection and protection, etc.

The process of layer-by-layer (LbL) self-assembly has been used to construct ultra thin films by alternately adsorbing onto a surface different components of a layered composition. For example, the different layers may comprise oppositely charged polyanions and polycations. The resulting films typically have had thicknesses in the nanometer range. Their permeability, solubility, morphology, and other characteristics may be modified according to the intended use.

LbL nanoassembled multilayers have been proposed for use as drug carrier systems. Typically, a central core containing the drug molecules is coated with a multilayer wall to act as a diffusion barrier. A typical drug release time has been 1˜4 hours. This process works best with drugs that do not aggregate, or otherwise lose potency, at high local concentrations.

C. Loo et al., “Immunotargeted nanoshells for integrated cancer imaging and therapy,” Nano Letters, vol. 5, pp. 709-711 (2005) discloses the synthesis of nanoshells having a dielectric silica core surrounded by a thin gold shell. By controlling the dimensions of the components, the optical properties of the nanoshells could be altered. The authors suggested that antibodies or other targeting moieties might be conjugated to the surface of the gold shell, e.g., via a sulfur-containing group such as a thiol; and that the nanoshells might then be used to target cancer cells for imaging and therapy.

Y. Lvov et al., “Biocolloids with ordered urease multilayer shells as enzymatic reactors,” Anal. Chem., vol. 73, pp. 4212-4217 (2001) discloses the layer-by-layer assembly of shells containing the enzyme urease onto 470 nm diameter latex spheres, and the use of the particles in catalysis.

N. Pargaonkar et al., “Controlled release of dexamethasone from microcapsules produced by polyelectrolyte layer-by-layer nanoassembly,” Pharm. Res., vol. 22, pp. 826-835 (2005) discloses the layer-by-layer assembly of particles having a dexamethasone microcrystal core. The core was encapsulated by multiple bilayers of alternating positively-charged poly (dimethyldiallyl ammonium chloride), and negatively charged sodium poly(styrenesulfonate). Dexamethasone is a hydrophobic glucocorticoid that is insoluble in water, and that has anti-inflammatory and immunosuppressive effects. The poly (dimethyldiallyl ammonium chloride) and sodium poly(styrenesulfonate) do not have substantial pharmacological activity themselves, but instead acted to encapsulate the pharmacologically active dexamethasone core.

The so-called “lytic peptides” occur naturally in a number of species, and many synthetic lytic peptide analogs have also been reported. Lytic peptides are linear; they are positively charged at physiological pH; they assume an amphipathic, α-helical conformation in a hydrophobic environment such as a phospholipid membrane; and they rapidly destroy negatively-charged phospholipid membranes when they are present in sufficient concentration. Ligand-lytic peptide conjugates have proven to be very potent in destroying tumors and metastases in vivo. We and our colleagues have previously shown that conjugates of lytic peptides (e.g., hecate or Phor14) with a 15 amino acid segment of the beta chain of human chorionic gonadotropin or luteinizing hormone (hCG/LH) are capable of targeting and destroying prostate, ovarian, and breast cancer cells, all of which express LH/CG receptors in vitro and in vivo. See C. Leuschner et al., “Targeted destruction of androgen-sensitive and -insensitive prostate cancer cells and xenografts through luteinizing hormone receptors,” The Prostate, vol. 46, pp. 116-125 (2001). See also U.S. Pat. No. 6,635,740. The toxicity of the conjugates against each of these cancer cell types depends directly upon hCG/LH receptor expression. However, the conjugates have a short half-life in circulation, and generally require multiple injections to completely eradicate tumors. See C. Leuschner et al, “Conjugates of lytic peptides target and destroy prostate cancer metastases,” in 16th EORTC-NCl-AACR Symposium, EJC Supplements, Abstract 75, p. 26 (Geneva, 2004). The in vivo efficacy of treatment in breast cancer xenograft-bearing mice depended on the concentration of intravenously injected Phor21-βCG(ala). Cell death in treated tumors was significantly higher in treatment groups receiving concentrations of 0.2 and 2 mg/kg body weight groups (p<0.05). Paradoxically, cell death was lower for treatments of 8 mg/kg body weight groups (p<0.01), an effect that we attributed to peptide aggregation followed by inactivation. See C. Leuschner et al, “A Comparison of the Toxicities and Side Effects of Conjugates of CG and Lytic Peptides,” Abstract LB-272, p. 118, 95th Annual Meeting of the American Association for Cancer Research, Orlando, Fla. (2004).

Other toxins have also been used in hormone-toxin cytotoxic conjugates used to target cancer cells selectively. See, e.g., A. Nagy et al., “Targeting cytotoxic conjugates of somatostatin, luteinizing hormone-releasing hormone and bombesin to cancers expressing their receptors: A ‘smarter’ chemotherapy,” Curr. Pharm. Design., vol. 11, pp. 1167-1180 (2005)

Peptides are quickly degraded in biological environments, e.g., by proteolysis. For example, luteinizing hormone releasing hormone (LHRH) has a half-life of ˜20 minutes in vivo. This observation has prompted some workers to design more stable analogs (i.e. Leuprolide).

U.S. patent application Ser. No. 10/816,732 discloses compositions and methods for the targeted and controlled release of substances such as drugs using magnetic nanoparticles encapsulated in a polymer. The compositions and methods may also be used to enhance imaging of tissues.

International patent application WO 2007/021621 discloses the use of magnetic nanoparticles that are covalently bound to a ligand to enhance imaging of tissues, or to selectively deliver drugs to cells.

There is an unfilled need for improved compositions and methods for the sustained release of small molecules, such as the release of pharmaceutical compounds in vivo, for example ligand-lytic peptide conjugates; particularly for molecules that may self-aggregate, or that otherwise become less effective at higher concentrations, or that half a short half-life in circulation. (Lytic peptides typically have a half-life in plasma of only ˜1-4 hours.) Because prior methods and compositions for the controlled release of molecules typically have a synthetic step during which the molecule is present in high concentrations, or employ compositions in which the encapsulated molecules have high local concentrations, the prior methods and compositions are subject to limitations imposed by self-aggregation, or by inactivation of the molecules, e.g., by proteolysis in a biological environment.

### DISCLOSURE OF THE INVENTION

We have discovered improved nanoparticle compositions and methods for the sustained release of small molecules, such as the release of pharmaceutical compounds in vivo, for example ligand-lytic peptide conjugates. Examples particularly include but are not limited to molecules that may self-aggregate or otherwise become less effective in higher concentrations or under physiological conditions, such as some of the ligand-lytic peptide conjugates and other peptide pharmaceuticals. The construction of the novel nanoparticles helps to prevent self-aggregation of the molecules, and to prevent loss of effectiveness through proteolysis in a biological environment. The novel system employs layer-by-layer self-assembly of biocompatible polyelectrolyte layers, and layers of charged small molecules such as drug molecules, particularly charged peptides, to form a multilayer nanoparticle in which the drug (or other small molecule) itself acts as one of the alternating charged layers in the multilayer assembly. The small molecules can then be released over time in a sustained manner. The LbL nano-assemblies can specifically target cancers, metastases, or other diseased tissues, can avoid RES uptake, can avoid accumulation in the liver, spleen, and bone marrow. Optionally, superparamagnetic nanoparticles may be incorporated to facilitate imaging of the tissues that are selectively targeted by the particles.

The novel system avoids the need for bolus injection of small molecules; it allows one to protect small molecules from degradation in circulation; it helps avoid deactivation by aggregation of the small molecules; it facilitates controlled and sustained release; it decreases systemic exposure and side effects from released molecules; and it decreases the effects of degradation in a biological environment. The nanosized materials can pass directly into diseased tissues and even directly into cells. Furthermore, optional ligand conjugation facilitates long circulation times and target recognition, endocytotic uptake by or accumulation on the membranes of target cells, and masking from RES, macrophages, and the immune system generally.

The process of preparation the novel nanoparticles can be relatively easy to implement. Precise amounts of a particular molecule, such as a drug, may be released over a long term. Preparation is preferably carried out under mild, aqueous conditions. The polyelectrolyte layers act as a storage device, and can help inhibit degradation of the “payload” molecules, for example, by inhibiting proteolysis of peptide drugs. Also, one can avoid high concentrations of the payload molecule in solution, which is advantageous where higher concentrations can lead to deactivation of the payload or where higher concentrations are otherwise undesirable. For example, our laboratory has found that increased concentrations of the anti-cancer peptide Phor21-βCG(ala) can actually reduce potency against targeted cancers and metastases. This effect can be avoided through the use of the present invention. Where some prior work has focused on designing more stable analogs, the novel approach instead allows one to embed a sensitive peptide (or other compound) in LbL nanoparticles to promote their slow release and a more consistent systemic concentration of the compound. Some compounds with potential medical uses can be highly toxic. Embedding such compounds in accordance with the present invention can help to reduce the toxic effects that can follow from bolus injections.

Prior work with drug-containing nanoparticles has focused primarily on encapsulating the drug molecules, incorporating the encapsulated molecules into multilayers formed of other components. Little (if any) attention has previously been given to incorporating drug molecules as an intrinsic component of a multilayer assembly. The novel approach alternates layers of charged drug molecules with layers of oppositely charged polymers. This approach allows one to avoid the preparation of a highly concentrated drug suspension, which can be problematic. The drug molecules, tightly bound within polyelectrolyte multilayers, may be released slowly, in a sustained fashion, retaining their biological activity over extended times.

In a prototype embodiment, we used silica nanocores with Phor21-βCG(ala) drug molecules and polyanions polyanions such as gelatin B or carboxymethylcellulose in multilayer nanoshells. We used the membrane-disrupting peptide Phor21, which we have found to be more potent in destroying cancer cells than either Hecate or Phor14. The Phor21-βCG(ala) conjugate peptide contains 35 amino acid residues: KFAKFAKKFAKFAKKFAKFAK-SYAVASAQAALAAR (SEQ ID NO. 1). The amino end of the peptide, residues 1-21, is the lytic peptide Phor21. The carboxy end of the peptide, residues 22-35, is a gonadotropin analog ligand, βCG(ala). The βCG(ala) ligand increases the selectivity of the conjugate towards cells with receptors for CG or LH. In the βCG(ala) fragment, cysteines from the native sequence were replaced by alanines, which increased our synthetic yield. The calculated isoelectric point of the peptide conjugate was 11.4; i.e., the peptide is positively charged at physiological pH. This positive charge is used directly in preparing the layer-by-layer assemblies with negatively-charged polyanions.

Multilayer decomposition and peptide release occurred with characteristic times of 20-30 hours. In vitro drug activity studies in a human breast cancer cell line showed high activity against human tumor cells. Encapsulation and sustained release of the drug increased treatment efficacy. Without wishing to be bound by these hypotheses, we believe that the enhanced efficacy resulted primarily from two factors: (1) inhibiting proteolytic degradation of the peptide, and (2) inhibiting inactivation by peptide aggregation at higher concentrations.

FIG. 1 depicts schematically the assembly of the nanoparticles (left), the assembled nanoparticles (center), and the release of drug from the nanoparticles (right). The large spheres in FIG. 1 denote the cores, e.g., silica; the small ellipses denote the drug, e.g., ligand-lytic peptide conjugate; and the wavy lines denote the polyanions.

The novel system for delivering ligand-lytic peptide conjugates has several advantages over current chemotherapy approaches. These advantages include high specificity and selectivity for target cells such as tumor cells and metastases; minimal side effects; minimal effect on the immune system; easy administration of nanometer-sized particles; easy access to tumor tissue and metastases; and avoiding bolus injection of drug molecules at high concentration. Other advantages include prolonged stability of the injected drug; increased efficacy and efficiency of the drug; and reduction in the total amount of drug needed to treat conditions such as primary tumors and metastases. As one example, the invention may be used to substantially enhance the ability to treat cancers and their metastases by combining the unique capabilities of lytic peptides to destroy cancer cells, irrespective of proliferation rates, and nanotechnology approaches for sustained drug release. For example, the following cancers all express LHRH receptors, and could be treated with compositions in accordance with the present invention, using LHRH as the ligand: prostate, ovary, breast, pancreas, testis, melanoma, colon, rectum, non-Hodgkins lymphoma, brain, oral pharynx, and endometrium. LH or CG receptors are expressed in all of the above cancers, as well as in lung and bladder cancers. Metastases of these cancers generally over-express both receptors. The encapsulation and sustained release of these peptide conjugates can also help reduce systemic toxicity from exposure at high dosages.

### BRIEF DESCRIPTION OF THE DRAWINGS

FIG. 1 depicts schematically the assembly of the nanoparticles (left), the assembled nanoparticles (center), and the release of drug from the nanoparticles (right).

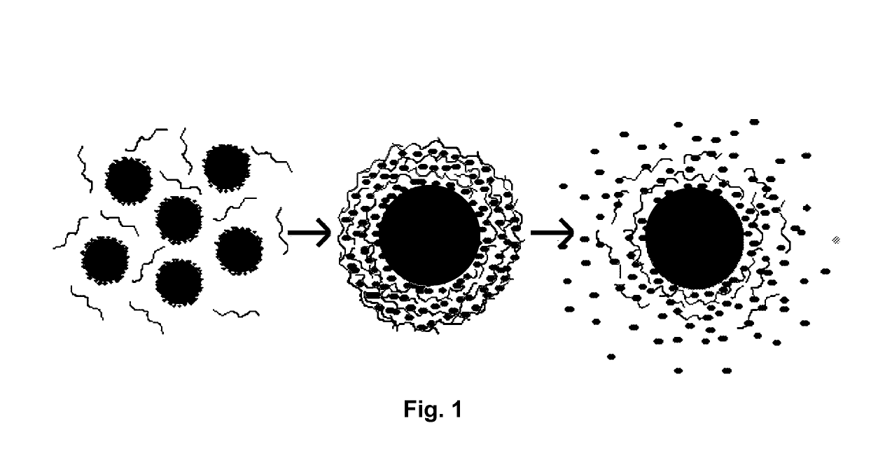

FIGS. 2(a) and (b) depict how the 4-potential of the particles changed with the adsorption of each additional polyelectrolyte layer.

FIG. 3(a) depicts the increasing mass of the particles during layer-by-layer assembly as measured by QCM. FIG. 3(b) depicts peptide concentration in the supernatant, as measured by UV absorbance.

FIG. 4 depicts the total amount of peptide released as a function of time from 20-bilayer-coated slides at two different pH values.

FIG. 5 depicts the total amount of peptide released as a function of time from 4-bilayer- and 8-bilayer-coated nanoparticles.

FIG. 6 depicts the toxicity of the Phor21-βCG(ala) nanoshells, of various controls, and of the free Phor21-βCG(ala) peptide against breast cancer cells in vitro.

### MODES FOR CARRYING OUT THE INVENTION

### Example 1

### Materials

Sodium carboxymethylcellulose (CMC) (MW 90,000) and gelatin from bovine skin, type B (Gelatin B. MW 20,000-25,000) were purchased from Sigma-Aldrich. The anti-cancer lytic polypeptide Phor21-βCG(ala) (MW 4,010) was obtained in lyophilized form from the National Cancer Institute (Bethesda, Md.). Silica nanoparticles (diameter 450 nm+30 nm) were purchased from Polysciences Inc. in 5.7% aqueous dispersion. The release medium used in these experiments was 0.9% sodium chloride, injectable USP solution (B. Braun Medical Inc., pH 5.6). The human breast cancer cell line MDA-MB-435S was obtained from the American Type Culture Collection (Rockville, Md.). Thiazolyl Blue was obtained from Sigma-Aldrich. All materials were used as received, unless otherwise noted. Although 450 nm diameter silica cores were used in the prototypes, larger or smaller particles may also be used without otherwise changing the techniques described, except that in general a smaller diameter core will require either a higher centrifugation speed or a longer centrifugation time at the sample separation stage.

### Example 2

### Preparation of Silica-Polyanion-Peptide Core-Shell Nanoparticles

Polyelectrolyte multilayers were deposited on silica nanoparticles using procedures generally following those of M. McShane et al., “Layer-by-Layer Electrostatic Self-Assembly, pp. 1-20 in J. Schwartz (ed.), Dekker Encyclopedia of Nanoscience and Nanotechnology (2004); and Y. Lvov et al., “Assembly of Multicomponent Protein Films by Means of Electrostatic Layer-by-Layer Adsorption,” J. Am. Chem. Soc., vol. 117, pp. 6117-6123 (1995). The CMC or gelatin B was negatively charged, and the Phor21-βCG(ala) in deionized (DI) water was positively charged. Typically, CMC or gelatin B (0.5 mL of a 2 mg/mL solution in 0.2 M aqueous NaCl) and Phor21-βCG(ala) (0.5 mL of a 1 mg/mL solution in 0.2 M aqueous NaCl) were added alternately into 1.5 mL silica particle suspensions (20 mg silica total mass). The adsorption of each polyelectrolyte or peptide layer was complete within 30 min at 4° C. Between depositions of successive layers, the particles were washed with DI water at 4° C., with centrifugation at 2,000 rpm for 10 min. Either four or eight bilayers were thus coated onto the silica particles. Additional layers may also be added by repeating the deposition and washing steps in the same manner. After the desired number of layers was deposited, the assembled core-shell nanoparticles were either lyophilized or stored at −20° C. until used.

### Example 3

### Characterization of Silica-Polyanion-Peptide Core-Shell Nanoparticles by QCM and by Surface Charge

The assembly of layers onto the silica nanocores was confirmed by monitoring Quartz Crystal Microbalance resonance frequency changes (QCM, USI-Systems, Japan), and also by observing changes in the electrophoretic potential (4-potential) after the deposition of each layer using a Zeta Potential Analyzer (Brookhaven Instruments Corporation).

### Example 4

### Characterization of Silica-Polyanion-Peptide Core-Shell Nanoparticles by UV-Vis Absorbance

The amount of peptide adsorbed onto the nanoparticles was monitored by UV-Vis absorbance (Agilent model 8543). After adsorption of peptide onto the cores, the particles were centrifuged as previously described. The supernatant was collected, and centrifuged again at 5,000 rpm for an additional 10 min. Absorbance of the supernatant was measured at 281 nm. The amount of Phor21-βCG(ala) adsorbed onto the cores was then calculated as the difference between the original amount of peptide in solution, compared with the amount of peptide that remained in the supernatant.

### Example 5

### Characterization of Silica-Polyanion-Peptide Core-Shell Nanoparticles by CLSM

The LbL assembly was further examined by confocal laser scanning microscopy (CLSM, Leica TCS SP2). Prior to LbL assembly as otherwise described above, the peptide was labeled with rhodamine β isothiocyanate (RBITC, Sigma-Aldrich) by dialysis for 72 hours at 4° C. in DI water. The peptide-silica nanoshells were visualized with a 63× objective lens at a 525 nm excitation wavelength.

### Example 6

### Release Kinetics In Vitro from Glass Slides

Peptide release was initially evaluated using negatively-charged, planar glass slides as a model for the nanoparticles. Twenty bilayers of peptide and CMC were alternately coated onto glass slides, following the same general procedures otherwise described above for preparing layered nanoparticles, without centrifugation. The initial and final peptide concentrations were measured by UV-Vis absorption; and the amount of peptide adsorbed onto the slides was calculated from the observed differences. Release kinetics were measured at 37° C. at two different pH values: 0.01 M acetic acid buffer with 0.9% NaCl (pH 4.5), and 0.9% NaCl USP injection solution (pH 5.6). The peptide-coated glass slides were immersed in the release media with stirring at 800 rpm. The UV absorbance of the release media was measured, and the percentage of peptide released was calculated from those measurements as a function of time.

### Example 7

### Release Kinetics In Vitro from Nanoparticles

Peptide release was also measured from the core-shell nanoparticles, following generally similar procedures. 20 mg of silica nanoparticles (15 mg/mL) were coated with a certain number of peptide layers and added to 2.0 mL release buffer in a centrifuge tube (in a 37° C. water bath) with continuous stirring at 800 rpm. At certain time intervals, a 0.5 mL particle suspension was removed and centrifuged at 5,000 rpm for 10 min. UV absorbance of the supernatant was measured at 281 nm. Following the UV measurement, the supernatant and pellet were mixed back into the original suspension.

### Example 8

### Cytotoxicity Studies

MDA-MB-435S cells were seeded into 12 well plates and grown in culture media containing Leibovitz's L-15 medium, 10% fetal bovine serum, 0.01 mg/mL bovine insulin, 100 IU/mL penicillin, and 100 μg/mL streptomycin. The cells were kept in tightly closed flasks at 37° C. in an incubator and grown to 70% confluence. Treatments with free Phor21-βCG(ala) were conducted at concentrations of 5, 20, and 100 μM. Treatments with silica-peptide nanoshells were conducted at corresponding amounts, equivalent to total Phor21-βCG(ala) concentrations of 5, 20, and 100 μM. On a separate plate silica nanoshells without peptide (15 mg/mL culture medium) were added to MDA-MB-435S cell cultures to determine their effect on cell viability. In all incubations, added saline was used as a control. Total incubation time was 9 hours at 37° C. Cell viability was measured in a thiazolyl Blue assay using [3-(4,5)-Dimethylthiazol-2-yl]-2,5 Diphenyltetrazolium Bromide—MTT. Cleavage of the tetrazolium ring by active mitochondria was used as a measure of the number of living cells. Statistical significance was determined using analysis of variance (ANOVA) and a two-tailed Student's t-test. Differences were considered significant at p<0.05.

### Example 9

### ζ-Potential of Nanoparticle Assemblies

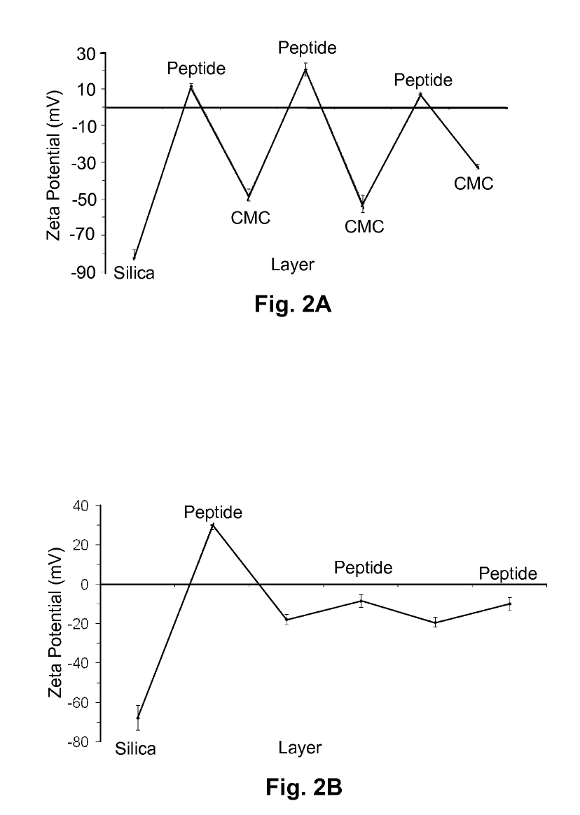

FIGS. 2(a) and (b) depict how the ζ-potential of the particles changed with the adsorption of each additional polyelectrolyte layer, for assemblies with CMC in FIG. 2(a), and with Gelatin B in FIG. 2(b). After initial washing, the silica nanoparticles had a ζ-potential of −70±10 mV. After adsorption of the cationic peptide, the surface potential increased to +20±3 mV. After deposition of anionic CMC, the surface potential decreased to −48±4 mV. This pattern repeated with the deposition of subsequent layers. The surface charge reversed with each successive layer. A generally similar pattern was seen with alternating adsorption of Phor21-βCG(ala) and gelatin B layers. However, with the Gelatin B, the surface charge did not totally reverse when a peptide layer was adsorbed, instead increasing to about −10 mV. At least for the Phor21-βCG(ala) peptide, Gelatin B is a preferred material for the polyanion layers.

### Example 10

### Mass and Thickness of Layers as Measured by QCM

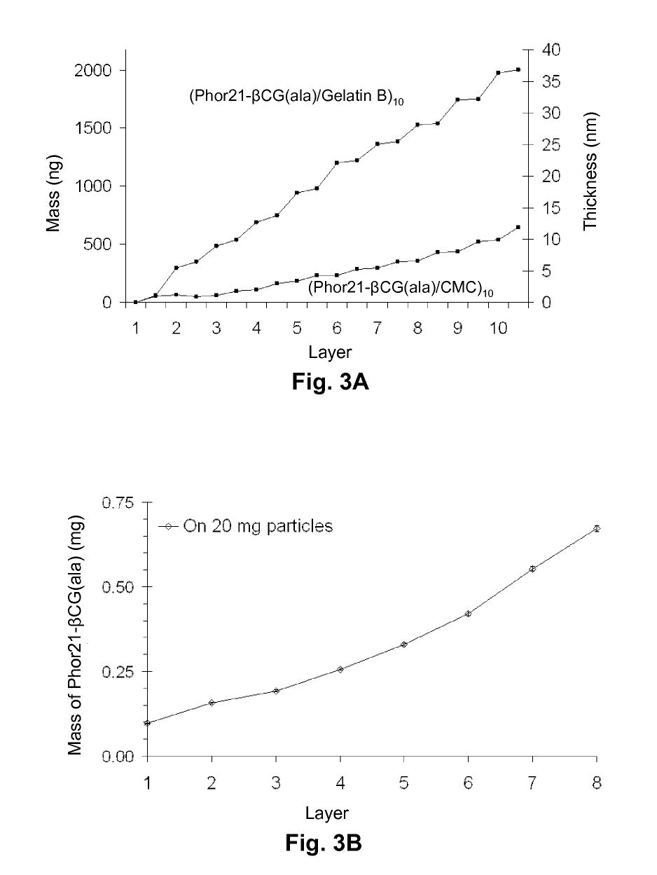

FIG. 3(a) depicts the increasing mass of the particles during layer-by-layer assembly with the two polyanions CMC and Gelatin B, as measured by QCM. The QCM observations showed a stable growth of Phor21-βCG(ala) layers alternating with polyanions. The increase in mass as successive layers were added was approximately linear. The average thickness of a peptide/polyanion bilayer was estimated as 0.8±0.2 nm using the Sauerbrey equation. The mass of one peptide layer on a 20 mg silica nanoshell corresponded to ca. 0.10±0.02 mg using the QCM data at an adsorption efficacy of 20%. One can see from FIG. 3(a) that the frequency decrease was sharper for peptide/gelatin B bilayers than for peptide/CMC bilayers. The average thickness of the bilayers was similar for both polyanions; the average thickness of bilayers was ca. 0.72±0.37 nm (CMC) and 0.81±0.18 nm (gelatin B) with estimated peptide amounts of 0.09-0.1 mg Phor21-βCG(ala) per 20 mg of silica shells per bilayer. The substantial frequency decrease was therefore attributed to differential adsorption of the polyanion. The calculated amount of peptide adsorbed was 0.40±0.09 mg for four-peptide-layer coatings and 0.81±0.18 mg for eight-peptide-layer coatings.

### Example 11

### Accretion of Layers as Measured by UV Absorption

FIG. 3(b) depicts peptide concentration in the supernatant, as measured by UV absorbance at 281 nm. The amount of peptide adsorbed onto 20 mg of silica nanoparticles alternated with CMC was calculated as ca. 0.26±0.01 mg of peptide for a four-bilayer coating, and ca. 0.67±0.01 mg of peptide for an eight-bilayer coating. The results from the QCM measurements were about 20-50% higher than those from the UV measurements. The UV results showed an exponential growth in the mass of peptide adsorbed, while the QCM results were more linear. Without wishing to be bound by this hypothesis, these differences in measurements may have resulted from the centrifugation and strong vortexing applied to wash and resuspend the particles.

### Example 12

### Confocal Microscopy Image Analysis

Confocal microscopy images (not shown) indicated that the silica nanoparticles were essentially totally coated with peptide, and that the nanoparticle size had increased due to the coatings. There were some slight aggregations of nanoparticles, but most were well separated from one another in DI water.

### Example 13

### Release of Peptide from Glass Slides

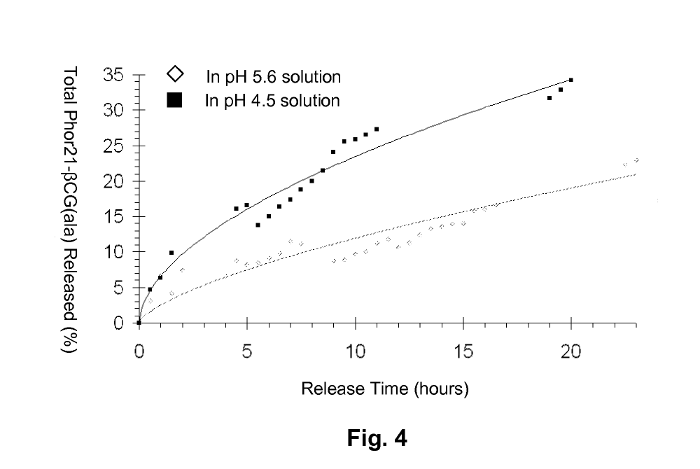

We first measured the kinetics of release of the Phor21-βCG(ala) peptide from multilayer assemblies on peptide-coated glass slides. FIG. 4 depicts the total amount of peptide released as a function of time from 20-bilayer-coated slides at two different pH values. After 20 hours, about 34% of the peptide had been released at pH 4.5 (closed boxes). After 23 hours, about 23% of the peptide had been released at pH 5.6 (open diamonds).

### Example 14

### Release of Peptide from Nanoshells

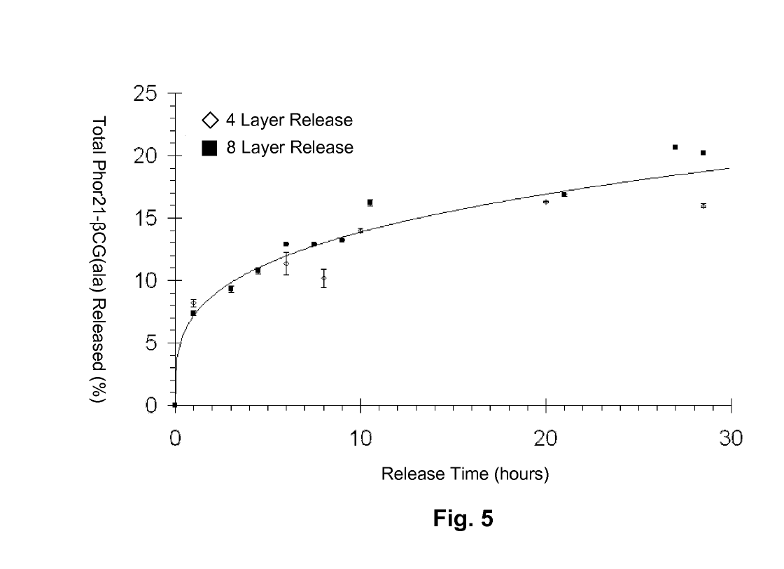

We then measured the kinetics of release of the Phor21-βCG(ala) peptide from multilayer assemblies on silica nanoshells. FIG. 5 depicts the total amount of peptide released as a function of time from 4-bilayer-coated nanoparticles (open diamonds) and 8-bilayer-coated nanoparticles (closed boxes). A 0.9% sodium chloride injection USP solution was used as the model in vitro release media, as it will maintain the activity of the peptide, is biocompatible, non-pyrogenic, and may likewise be used for in vivo studies. For both 4-layer and 8-layer peptide coatings, about 18% of the peptide was released after 28 hours. The peptide release rates from both the slides and nanoparticles followed an exponential trend, i.e., first-order kinetics. The data showed only minor differences between the release kinetics of peptides from four-layer and eight-layer nanoshells. Both assemblies released Phor21-βCG(ala) from the LbL multilayers relatively slowly. Extrapolating the observed release data curves predicts that ca. 50% total release will occur in about 7 days. The ionization fraction of the CMC carboxyl group is smaller at lower pH values, thereby weakening the interaction between polyelectrolyte molecules, and perhaps accounting for the faster release of peptide at the lower pH.

### Example 15

### In Vitro Toxicity against Breast Cancer Cells

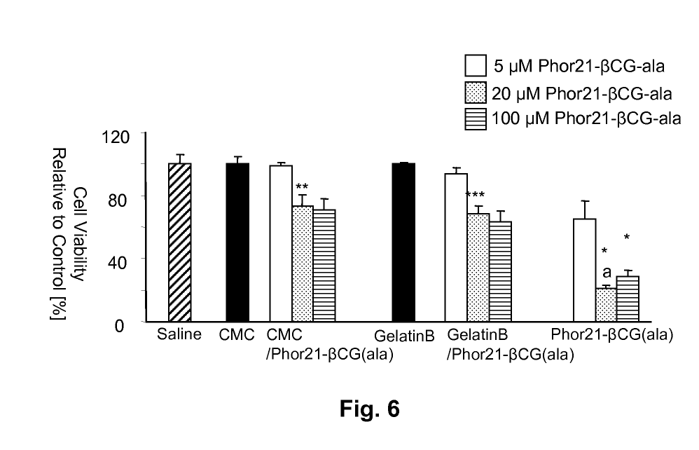

FIG. 6 depicts the toxicity of the Phor21-βCG(ala) nanoshells, of various controls, and of the free Phor21-βCG(ala) peptide against MDA-MB-435S human breast cancer cells in vitro over an incubation period of 9 hours (N=6). In FIG. 6: The symbol * denotes significantly different, p<0.0001; compared to saline controls, compared to peptide-silica nanoshells (CMC), and (Gelatin B) at 20 and 100 μM of Phor21-βCG(ala). The symbol ** denotes significantly different, p<0.026; CMC peptide-silica nanoshells: 5 versus 20 μM. The symbol *** denotes significantly different, p<0.0035; Gelatin B peptide-silica nanoshells: 5 versus 20 μM. The symbol “a” denotes significantly different, p<0.012; compared to 100 μM. The cancer cells were destroyed by free Phor21-βCG(ala), although the effectiveness decreased with increasing concentration at 100 μM (p<0.012), which suggested deactivation of the Phor21-βCG(ala) peptide through aggregation. Free peptide administered at 100 micromolar showed significantly lower activity than at 20 micromolar, presumably due to aggregation. By contrast, there was no activity loss at the 100 micromolar level versus 20 micromolar for the LbL nanoparticles. It has previously been shown that lytic peptide-βCG conjugates have low toxicity towards cells that do not express the CG receptor. See C. Leuschner et al., “Targeted destruction of androgen-sensitive and insensitive prostate cancer cells and xenografts through luteinizing hormone receptors,” The Prostate, vol. 46, pp. 116-125 (2001).

Embedding the Phor21-βCG(ala) in nanoparticles facilitated the steady release of the compound. The free peptide is almost completely destroyed in circulation after about three hours. By contrast, the concentration of released peptide from the LBL nanoparticles held at levels about 3.2-16 micromolar over a period of 9 hours. These concentrations were lower than from a bolus administration of 20 or 100 micromolar, but the toxicity was comparable to 5 micromolar free peptide concentrations.

Conjugates Useful in the Present Invention

This invention may be practiced with a variety of small charged molecules, preferably small charged molecules with pharmaceutical activity, most preferably with ligand-lytic peptide conjugates, wherein the ligand is a hormone or hormone analog with specificity for the target cells, and the lytic peptide is toxic to the target cells. Examples of such ligand-lytic peptide conjugates are disclosed and discussed extensively, for example, in U.S. Pat. No. 6,635,74.

Lytic Peptides Useful in the Present Invention

It is believed (without wishing to be bound by this theory) that cationic amphipathic peptides act by disrupting negatively-charged cell membranes. It is believed that tumor cells tend to have negatively-charged membranes, compared to more neutral membranes for normal mammalian cells, and are thus more susceptible to disruption by cationic amphipathic peptides. With ligand-lytic peptide conjugates, cell death results from the increased effective concentration of lytic peptide in the vicinity of cells with corresponding receptors, or internalization of lytic peptide into such cells, or both.

Although the embodiments of this invention that have been tested to date have used Phor21 as the effector lytic peptide, this invention will work with a combination of a ligand with other lytic peptides as well. The so-called Phor peptides, for example, are disclosed in M. Javadpour et al., “Self Assembly of Designed Antimicrobial Peptides in Solution and Micelles,” Biochem., vol. 36, pp. 9540-9549 (1997). Many lytic peptides are known in the art and include, for example, those mentioned in the references cited in the following discussion.

Lytic peptides are small, cationic peptides. Native lytic peptides appear to be major components of the antimicrobial defense systems of a number of animal species, including those of insects, amphibians, and mammals. They typically comprise 23-39 amino acids, although they can be smaller. They have the potential for forming amphipathic alpha-helices. See Boman et al., “Humoral immunity in Cecropia pupae,” Curr. Top. Microbiol. Immunol. vol. 94/95, pp. 75-91 (1981); Boman et al., “Cell-free immunity in insects,” Annu. Rev. Microbiol., vol. 41, pp. 103-126 (1987); Zasloff, “Magainins, a class of antimicrobial peptides from Xenopus skin: isolation, characterization of two active forms, and partial DNA sequence of a precursor,” Proc. Natl. Acad. Sci. USA, vol. 84, pp. 3628-3632 (1987); Ganz et al., “Defensins natural peptide antibiotics of human neutrophils,” J. Clin. Invest., vol. 76, pp. 1427-1435 (1985); and Lee et al., “Antibacterial peptides from pig intestine: isolation of a mammalian cecropin,” Proc. Natl. Acad. Sci. USA, vol. 86, pp. 9159-9162 (1989).

Known amino acid sequences for lytic peptides may be modified to create new peptides that would also be expected to have lytic activity by substitutions of amino acid residues that promote alpha-helical stability and that preserve the amphipathic nature of the peptides (e.g., replacing a polar residue with another polar residue, or a non-polar residue with another non-polar residue, etc.); by substitutions that preserve the charge distribution (e.g., replacing an acidic residue with another acidic residue, or a basic residue with another basic residue, etc.); or by lengthening or shortening the amino acid sequence while preserving its amphipathic character or its charge distribution. Lytic peptides and their sequences are disclosed in Yamada et al., “Production of recombinant sarcotoxin IA in Bombyx mori cells,” Biochem. J., vol. 272, pp. 633-666 (1990); Taniai et al., “Isolation and nucleotide sequence of cecropin B cDNA clones from the silkworm, Bombyx mori,” Biochimica Et Biophysica Acta, vol. 1132, pp. 203-206 (1992); Boman et al., “Antibacterial and antimalarial properties of peptides that are cecropin-melittin hybrids,” Febs Letters, vol. 259, pp. 103-106 (1989); Tessier et al., “Enhanced secretion from insect cells of a foreign protein fused to the honeybee melittin signal peptide,” Gene, vol. 98, pp. 177-183 (1991); Blondelle et al., “Hemolytic and antimicrobial activities of the twenty-four individual omission analogs of melittin,” Biochemistry, vol. 30, pp. 4671-4678 (1991); Andreu et al., “Shortened cecropin A-melittin hybrids. Significant size reduction retains potent antibiotic activity,” Febs Letters, vol. 296, pp. 190-194 (1992); Macias et al., “Bactericidal activity of magainin 2: use of lipopolysaccharide mutants,” Can. J. Microbiol., vol. 36, pp. 582-584 (1990); Rana et al., “Interactions between magainin-2 and Salmonella typhimurium outer membranes: effect of Lipopolysaccharide structure,” Biochemistry, vol. 30, pp. 5858-5866 (1991); Diamond et al., “Airway epithelial cells are the site of expression of a mammalian antimicrobial peptide gene,” Proc. Natl. Acad. Sci. USA, vol. 90, pp. 4596 ff (1993); Selsted et al., “Purification, primary structures and antibacterial activities of β-defensins, a new family of antimicrobial peptides from bovine neutrophils,” J. Biol. Chem., vol. 268, pp. 6641 ff (1993); Tang et al., “Characterization of the disulfide motif in BNBD-12, an antimicrobial β-defensin peptide from bovine neutrophils,” J. Biol. Chem., vol. 268, pp. 6649 ff (1993); Lehrer et al., Blood, vol. 76, pp. 2169-2181 (1990); Ganz et al., Sem. Resp. Infect. I., pp. 107-117 (1986); Kagan et al., Proc. Natl. Acad. Sci. USA, vol. 87, pp. 210-214 (1990); Wade et al., Proc. Natl. Acad. Sci. USA, vol. 87, pp. 4761-4765 (1990); Romeo et al., J. Biol. Chem., vol. 263, pp. 9573-9575 (1988); Jaynes et al., “Therapeutic Antimicrobial Polypeptides, Their Use and Methods for Preparation,” WO 89/00199 (1989); Jaynes, “Lytic Peptides, Use for Growth, Infection and Cancer,” WO 90/12866 (1990); Berkowitz, “Prophylaxis and Treatment of Adverse Oral Conditions with Biologically Active Peptides,” WO 93/01723 (1993).

Families of naturally-occurring lytic peptides include the cecropins, the defensins, the sarcotoxins, the melittins, and the magainins. Boman and coworkers in Sweden performed the original work on the humoral defense system of Hyalophora cecropia, the giant silk moth, to protect itself from bacterial infection. See Hultmark et al., “Insect immunity. Purification of three inducible bactericidal proteins from hemolymph of immunized pupae of Hyalophora cecropia,” Eur. J. Biochem., vol. 106, pp. 7-16 (1980); and Hultmark et al., “Insect immunity. Isolation and structure of cecropin D. and four minor antibacterial components from cecropia pupae,” Eur. J. Biochem., vol. 127, pp. 207-217 (1982).

Infection in H. cecropia induces the synthesis of specialized proteins capable of disrupting bacterial cell membranes, resulting in lysis and cell death. Among these specialized proteins are those known collectively as cecropins. The principal cecropins—cecropin A, cecropin B, and cecropin D—are small, highly homologous, basic peptides. In collaboration with Merrifield, Boman's group showed that the amino-terminal half of the various cecropins contains a sequence that will form an amphipathic alpha-helix. Andrequ et al., “N-terminal analogues of cecropin A: synthesis, antibacterial activity, and conformational properties,” Biochem., vol. 24, pp. 1683-1688 (1985). The carboxy-terminal half of the peptide comprises a hydrophobic tail. See also Boman et al., “Cell-free immunity in Cecropia,” Eur. J. Biochem., vol. 201, pp. 23-31 (1991).

A cecropin-like peptide has been isolated from porcine intestine. Lee et al., “Antibacterial peptides from pig intestine: isolation of a mammalian cecropin,” Proc. Natl. Acad. Sci. USA, vol. 86, pp. 9159-9162 (1989).

Cecropin peptides have been observed to kill a number of animal pathogens other than bacteria. See Jaynes et al., “In Vitro Cytocidal Effect of Novel Lytic Peptides on Plasmodium falciparum and Trypanosoma cruzi,” FASEB, 2878-2883 (1988); Arrowood et al., “Hemolytic properties of lytic peptides active against the sporozoites of Cryptosporidium parvum,” J. Protozool., vol. 38, No. 6, pp. 161S-163S (1991); and Arrowood et al., “In vitro activities of lytic peptides against the sporozoites of Cryptosporidium parvum,” Antimicrob. Agents Chemother., vol. 35, pp. 224-227 (1991). However, normal mammalian cells do not appear to be adversely affected by cecropins, even at high concentrations. See Jaynes et al., “In vitro effect of lytic peptides on normal and transformed mammalian cell lines,” Peptide Research, vol. 2, No. 2, pp. 1-5 (1989); and Reed et al., “Enhanced in vitro growth of murine fibroblast cells and preimplantation embryos cultured in medium supplemented with an amphipathic peptide,” Mol. Reprod. Devel., vol. 31, No. 2, pp. 106-113 (1992).

Defensins, originally found in mammals, are small peptides containing six to eight cysteine residues. Ganz et al., “Defensins natural peptide antibiotics of human neutrophils,” J. Clin. Invest., vol. 76, pp. 1427-1435 (1985). Extracts from normal human neutrophils contain three defensin peptides: human neutrophil peptides HNP-1, HNP-2, and HNP-3. Defensin peptides have also been described in insects and higher plants. Dimarcq et al., “Insect immunity: expression of the two major inducible antibacterial peptides, defensin and diptericin, in Phormia terranvae,” EMBO J., vol. 9, pp. 2507-2515 (1990); Fisher et al., Proc. Natl. Acad. Sci. USA, vol. 84, pp. 3628-3632 (1987).

Slightly larger peptides called sarcotoxins have been purified from the fleshfly Sarcophaga peregrina. Okada et al., “Primary structure of sarcotoxin 1, an antibacterial protein induced in the hemolymph of Sarcophaga peregrina (flesh fly) larvae,” J. Biol. Chem., vol. 260, pp. 7174-7177 (1985). Although highly divergent from the cecropins and defensins, the sarcotoxins presumably have a similar antibiotic function.

Other lytic peptides have been found in amphibians. Gibson and collaborators isolated two peptides from the African clawed frog, Xenopus laevis, peptides which they named PGS and Gly¹⁰Lys²²PGS. Gibson et al., “Novel peptide fragments originating from PGLₐ and the caervlein and xenopsin precursors from Xenopus laevis,” J. Biol. Chem., vol. 261, pp. 5341-5349 (1986); and Givannini et al., “Biosynthesis and degradation of peptides derived from Xenopus laevis prohormones,” Biochem. J., vol. 243, pp. 113-120 (1987). Zasloff showed that the Xenopus-derived peptides have antimicrobial activity, and renamed them magainins. Zasloff, “Magainins, a class of antimicrobial peptides from Xenopus skin: isolation, characterization of two active forms, and partial DNA sequence of a precursor,” Proc. Natl. Acad. Sci. USA, vol. 84, pp. 3628-3632 (1987).

Synthesis of nonhomologous analogs of different classes of lytic peptides has been reported to reveal that a positively charged, amphipathic sequence containing at least 20 amino acids appeared to be a requirement for lytic activity in some classes of peptides. Shiba et al., “Structure-activity relationship of Lepidopteran, a self-defense peptide of Bombyx more,” Tetrahedron, vol. 44, No. 3, pp. 787-803 (1988). Other work has shown that smaller peptides can also be lytic. See McLaughlin et al., cited below.

Cecropins have been shown to target pathogens or compromised cells selectively, without affecting normal host cells. The synthetic lytic peptide known as S-1 (or Shiva 1) has been shown to destroy intracellular Brucella abortus-, Trypanosoma cruzi-, Cryptosporidium parvum-, and infectious bovine herpes virus I (IBR)-infected host cells, with little or no toxic effects on noninfected mammalian cells. See Jaynes et al., “In vitro effect of lytic peptides on normal and transformed mammalian cell lines,” Peptide Research, vol. 2, No. 2, pp. 1-5 (1989); Wood et al., “Toxicity of a Novel Antimicrobial Agent to Cattle and Hamster cells In vitro,” Proc. Ann. Amer. Soc. Anim. Sci., Utah State University, Logan, Utah. J. Anim. Sci. (Suppl. 1), vol. 65, p. 380 (1987); Arrowood et al., “Hemolytic properties of lytic peptides active against the sporozoites of Cryptosporidium parvum,” J. Protozool., vol. 38, No. 6, pp. 161S-163S (1991); Arrowood et al., “In vitro activities of lytic peptides against the sporozoites of Cryptosporidium parvum,” Antimicrob. Agents Chemother., vol. 35, pp. 224-227 (1991); and Reed et al., “Enhanced in vitro growth of murine fibroblast cells and preimplantation embryos cultured in medium supplemented with an amphipathic peptide,” Mol. Reprod. Devel., vol. 31, No. 2, pp. 106-113 (1992).

Morvan et al., “In vitro activity of the antimicrobial peptide magainin 1 against Bonamia ostreae, the intrahemocytic parasite of the flat oyster Ostrea edulis,” Mol. Mar. Biol., vol. 3, pp. 327-333 (1994) reports the in vitro use of a magainin to selectively reduce the viability of the parasite Bonamia ostreae at doses that did not affect cells of the flat oyster Ostrea edulis.

Also of interest are the synthetic peptides disclosed in U.S. Pat. Nos. 6,566,334 and 5,789,542, peptides that have lytic activity with as few as 10-14 amino acid residues. Also of interest are analogs that contain D-amino acids.

Lytic peptides such as are known generally in the art may be used in practicing the present inventions. Selective toxicity to ligand-bound cells is desirable, especially when the ligand and peptide are administered separately. Selective toxicity is less important when the ligand and peptide are linked to one another, because in that case the peptide is effectively concentrated in the immediate vicinity of cells having receptors for the ligand.

Hormones and Hormone Analogs Useful in the Present Invention

Hormones that may be used in a ligand-lytic peptide conjugate in accordance with this invention include those for which receptors are preferentially expressed by the cancer cells other diseased cells, or other cells that are being selectively targeted. For example, for a pituitary adenoma the hormone may be selected from the group consisting of gonadotropin-releasing hormone, lamprey III luteinizing hormone releasing hormone (I-LHRH-III), corticosteroid-releasing hormone, growth hormone-releasing hormone, vasoactive intestinal polypeptide, and pituitary adenylate cyclase activating peptide, and analogs of those hormones and peptides.

For a breast cancer the hormone may be selected from the group consisting of gonadotropin-releasing hormone, lamprey III luteinizing hormone releasing hormone (I-LHRH-III), the beta subunit of chorionic gonadotropin, beta chain of luteinizing hormone (bLH), and analogs of one of those hormones.

For an ovarian cancer the hormone may be selected from the group consisting of gonadotropin-releasing hormone, lamprey III luteinizing hormone releasing hormone (I-LHRH-III), the beta subunit of chorionic gonadotropin, beta chain of luteinizing hormone (bLH), and analogs of one of those hormones.

For an endometrial cancer the hormone may be selected from the group consisting of gonadotropin-releasing hormone, lamprey III luteinizing hormone releasing hormone (I-LHRH-III), the beta subunit of chorionic gonadotropin, beta chain of luteinizing hormone (bLH), and analogs of one of those hormones.

For a prostate cancer the hormone may be selected from the group consisting of gonadotropin-releasing hormone, the beta subunit of chorionic gonadotropin, lamprey III luteinizing hormone releasing hormone (I-LHRH-III), MSH, EGF, FSH, Her-2, transferring, folic acid, and analogs of one of those hormones.

For a testicular cancer the hormone may be selected from the group consisting of gonadotropin-releasing hormone, lamprey III luteinizing hormone releasing hormone (I-LHRH-III), the beta subunit of chorionic gonadotropin, or beta chain of luteinizing hormone (bLH), and analogs of one of those hormones.

Other ligands (or their analogs) and their cancer targets that may be used in practicing this invention include, for example, the following:

Somatostatin: pituitary adenomas, gastroenteropancreatic cancer, small cell lung cancer, prostate, colon, breast, lung, ovarian, renal cell carcinoma
Gastrin-releasing peptide: small cell lung cancer, pancreatic, gastric, prostate
Bombesin: prostate, renal, breast, endometrial, ovarian, pancreatic, thyroid, brain
Estrogen, androgens: gonadotroph cancers
Her-2, Her-3: breast, prostate, colon,
LHRH: prostate, colon urinary bladder, melanoma, non-Hodgkins lymphoma, kidney, leukemia, oral pharynx, pancreas, brain, breast, uterine corpus, ovary, thyroid
LH/CG or βLH/βCG: lung, prostate, melanoma, uterine corpus, breast, ovary, testicular
FSH: renal, prostate, breast
MSH: melanoma, breast, prostate
Folate: breast cancer, nasopharyngeal, colon cancer, hepatic

### Transferrin: Glioma

alphaᵥ-beta₃: vasculature
VEGF: vasculature
EGF: lung, colon, prostate, breast

Analogs

Analogs of these and other hormones and ligands are well-known in the art, and may also be used in practicing this invention. As is well known in the art, an analog is a compound with a structure that is similar to that of the “parent” compound, and that has similar or opposing metabolic effects. Analogs may act either as agonists, having a similar effect, or antagonists, having a blocking effect. Some of the many examples known in the art are cited below. Included among the analogs of a ligand are antibodies or antibody fragments against the receptor for that ligand. The following discussion gives a number of examples, but is by no means an exhaustive listing.

Analogs of Gonadotropin Releasing Hormone

S. Sealfon et al., “Molecular mechanisms of ligand interaction with the gonadotropin-releasing hormone receptor,” Endocrine Reviews, vol. 18, pp. 180-205 (1997) is a review paper that, among other things, discusses the apparent role of each of the individual amino acids in the GnRH decapeptide, and gives extensive guidance on the types of substitutions that may be made in analogs. See particularly pp. 184-191 of this paper, and the schematic summary shown in FIG. 8 on page 190.

A 1986 review paper, M. Karten et al., “Gonadotropin-releasing hormone analog design. Structure-function studies toward the development of agonists and antagonists: rationale and perspective,” Endocrine Reviews, vol. 7, pp. 44-66 (1986), described or gave citations to over 2000 GnRH analogs (p. 44, par. 1) that had been synthesized and characterized over two decades before the filing date of the present application.

S. Sealfon et al., “The gonadotrophin-releasing hormone receptor: structural determinants and regulatory control,” Human Reproduction Update, vol. 1, pp. 216-230 (1995) provides a review of contemporaneous knowledge of GnRH receptor structure and regulation of receptor expression. This review article mentions the fact that thousands of GnRH analogs have been synthesized and studied (p. 216).

M. Filicori, “Gonadotropin-releasing hormone agonists: a guide to use and selection,” Drugs, vol. 48, pp. 41-58 (1994) is a review article discussing a number of GnRH agonists, and examples of the types of modifications that may be used to make such agonists. Among the examples mentioned are replacement of the tenth amino acid (glycine) of the native GnRH sequence with an ethylamide residue; or the substitution of the sixth amino acid residue (glycine) with other more lipophilic D-amino acids such as D-Phe, D-Leu, or D-Trp; or the incorporation of more complex amino acids in position 6, such as D-Ser (t-Bu), D-His (Bzl), or D-NaI(2); or in position 10, such as aza-Gly; or the N-Me-Leu modification in position 7 (see pp. 42 and 43). These modifications were said to result in more hydrophobic compounds that were more stable than the native GnRH molecule, and thus to have higher receptor affinity and in vitro potency. In addition, the more hydrophobic GnRH agonists were said to be more resistant to enzyme degradation, and to bind more strongly to plasma proteins and body tissues, thus decreasing renal excretion and prolonging drug half-life. This review article also discusses various routes of administration and delivery systems known in the art.

Another review article is P. Conn et al., “Gonadotropin-releasing hormone and its analogues,” New Engl. J. Med., vol. 324, pp. 93-103 (1991). Several GnRH analogs are disclosed including, as shown in Table 1 on p. 95, the analogs decapeptyl, leuprolide, buserelin, nafarelin, deslorelin, and histrelin; and several additional analogs discussed on p. 99.

A. Nechushtan et al., “Adenocarcinoma cells are targeted by the new GnRH-PE₆₆ chimeric toxin through specific gonadotropin-releasing hormone binding sites,” J. Biol. Chem., vol. 298, pp. 11597-11603 (1997) discloses a 67 kDa chimeric fusion protein comprising a Pseudomonas-derived toxin bound to a GnRH analog in which tryptophan replaced glycine as the sixth amino acid; as well as the use of that fusion protein to prevent the growth of colon carcinoma xenografts in nude mice, and to kill various adenocarcinoma cells in vitro.

G. Emons et al., “Growth-inhibitory actions of analogues of luteinizing hormone releasing hormone on tumor cells,” Trends in Endocrinology and Metabolism, vol. 8, pp. 355-362 (1997) discloses that in vitro proliferation of two human ovarian cancer cell lines, and of two human endometrial cancer cell lines, was inhibited by the LHRH agonist triptorelin; and that in vitro proliferation of ovarian and endometrial cancer cell lines was also inhibited by the LHRH antagonist Cetrorelix; while against another ovarian cancer cell line the antagonist did not have this effect, although it partly blocked the antiproliferative effect of the agonist triptorelin. Antiproliferative effects of LHRH analogs against prostate cancer cell lines in vitro were also reported. This paper also reports that chronic administration of LHRH agonists inhibited ovarian or testicular function in a reversible manner.

M. Kovacs et al., “Recovery of pituitary function after treatment with a targeted cytotoxic analog of luteinizing hormone-releasing hormone,” Proc. Natl. Acad. Sci. USA, vol. 94, pp. 1420-1425 (1997) discloses the use of a doxorubicin derivative conjugated to the carrier agonist [D-Lys⁶] LHRH to reversibly (i.e., temporarily) inhibit gonadotrophic cells in the pituitary. It was also reported that this LHRH analog-toxin conjugate inhibited the growth of prostate tumors in rats.

J. Janovick et al., “Gonadotropin releasing hormone agonist provokes homologous receptor microaggregation: an early event in seven-transmembrane receptor mediated signaling,” Endocrinology, vol. 137, pp. 3602-3605 (1996) discloses certain experiments using the agonist D-Lys⁶-GnRH-lactoperoxidase conjugate, and others using the antagonist D-pGlu¹-D-Phe²-D-Trp³-D-Lys⁶-GnRH-lactoperoxidase conjugate.

C. Albano et al., “Comparison of different doses of gonadotropin-releasing hormone antagonist Cetrorelix during controlled ovarian hyperstimulation,” Fertility and Sterility, vol. 67, pp. 917-922 (1997) discloses experiments conducted with the GnRH antagonist Cetrorelix to determine the minimal effective dose to prevent premature LH surge in patients undergoing controlled ovarian hyperstimulation for assisted reproductive technologies.

L. Maclellan et al., “Superstimulation of ovarian follicular growth with FSH, oocyte recovery, and embryo production from Zebu (Bos indicus) calves: Effects of Treatment with a GnRH Agonist or Antagonist,” Theriogenology, vol. 49, pp. 1317-29 (1998) describes experiments in which a GnRH agonist (deslorelin) or a GnRH antagonist (cetrorelix) were administered to calves to determine whether altering plasma LH concentration would influence follicular response to FSH and oocyte development.

A. Qayum et al., “The effects of gonadotropin releasing hormone analogues in prostate cancer are mediated through specific tumour receptors,” Br. J. Cancer, vol. 62, pp. 96-99 (1990) discloses experiments investigating the use of the GnRH analog buserelin on prostate cancers.

A. Cornea et al., “Redistribution of G_{q/11}α in the pituitary gonadotrope in response to a gonadotropin-releasing hormone agonist,” Endocrinology, vol. 139, pp. 397-402 (1998) discloses studies on the effect of buserelin, a metabolically stable GnRH agonist, on the distribution of the α-subunit of the guanyl nucleotide binding protein subfamily G_{q/11}.

Analogs of the Beta Subunit of Luteinizing Hormone or Chorionic Gonadotropin

Luteinizing hormone and chorionic gonadotropin are structurally and functionally homologous peptides. See, e.g., J. Lin et al., “Increased expression of luteinizing hormone/human chorionic gonadotropin receptor gene in human endometrial carcinomas,” J. Clinical Endocrinology & Metabolism, vol. 79, pp. 1483-1491 (1994).

D. Morbeck et al., “A receptor binding site identified in the region 81-95 of the β-subunit of human luteinizing hormone (LH) and chorionic gonadotropin (hCG),” Molecular & Cellular Endocrinology, vol. 97, pp. 173-181 (1993) discloses experiments in which two series of overlapping peptides (each 15 residues in length), comprising the entire sequences of the β-subunits of human lutropin (LH) and chorionic gonadotropin (hCG), were used to identify all linear regions of the subunit that participate in the binding of the hormone to the receptor. The most potent inhibitor in a competitive binding assay was a peptide containing residues 81-95 of hCG. In addition, other regions that inhibited binding were identified. A third set of peptides was prepared in which each residue of the 81-95 hCG sequence was sequentially replaced by alanine, to identify the more important residues for binding. Five such residues were identified as being important to binding. In addition to identifying the 81-95 hCG sequence as itself being a useful analog, this detailed information would be very useful in designing analogs of the beta subunit of luteinizing hormone or of chorionic gonadotropin.

V. Garcia-Campayo et al., “Design of stable biologically active recombinant lutropin analogs,” Nature Biotechnology, vol. 15, pp. 663-667 (1997) describes the synthesis of a luteinizing hormone analog, in which the α and β subunits were fused through a linker. The analog was found to be biologically active, and to have significantly greater in vitro stability than the native heterodimer.

T. Sugahara et al., “Biosynthesis of a biologically active single peptide chain containing the human common a and chorionic gonadotropin D subunits in tandem,” Proc. Natl. Acad. Sci. USA, vol. 92, pp. 2041-2045 (1995) describes the production of a chimeric peptide, in which the a and D subunits of human chorionic gonadotropin were fused into a single polypeptide chain. The resulting molecule was found to be efficiently secreted, and to show increased activity both in vitro and in vivo.

D. Puett et al., “The tie that binds: Design of biologically active single-chain human chorionic gonadotropins and a gonadotropin-receptor complex using protein engineering,” Biol. Repro., vol. 58, pp. 1337-1342 (1998) is a review of numerous published papers concerning human chorionic gonadotropin and its analogs, including the effects of chemical modifications, synthetic peptides, limited proteolysis, protein engineering to produce hormone chimeras, site-directed mutagenesis, and specific amino acid residues.

Y. Han et al., “hCGP Residues 94-96 alter LH activity without appearing to make key receptor contacts,” Mol. Cell. Endocrin., vol. 124, pp. 151-161 (1996) describes the effects on LH activity of several particular amino acid substitutions in the beta subunit of LH (namely, at residues 94-96). Not only are numerous analogs specifically described in this paper, but this type of information provides important guidance to one of skill in the art in designing other analogs.

Z. Zalesky et al, “Ovine luteinizing hormone: Isoforms in the pituitary during the follicular and luteal phases of the estrous cycle and during anestrus,” J. Anim. Sci., vol. 70, pp. 3851-3856 (1992) discloses thirteen isoforms of LH in ewes. Each of these thirteen isoforms could be considered an analog of LH.

A. Hartee, “Multiple forms of pituitary and placental gonadotropins,” pp. 147-154 in S. Milligan (Ed.), Oxford Reviews of Reproductive Biology (1989) discloses different glycoprotein variants that may be considered analogs of FSH, LH, and CG. Seven isoforms of LH, and six isoforms of hCG were isolated; all had bioactivity in vivo.

Follicle Stimulating Hormone

P. Grasso et al., “In vivo effects of follicle-stimulating hormone-related synthetic peptides on the mouse estrous cycle,” Endocrinology, vol. 137, pp. 5370-5375 (1996) discloses a synthetic tetrapeptide amide analog to the beta subunit of FSH, and its antagonistic effects both in vitro and in vivo.

J. Dias et al, “Human follicle-stimulating hormone structure-activity relationships,” Biol. Repro., vol. 58, pp. 1331-1336 (1998) is a review of numerous publications concerning human follicle stimulating hormone, structure-activity relationships, and FSH analogs, including the effects of glycosylation, synthetic peptides, site-directed mutagenesis, and specific amino acid residues.

A. Cerpa-Poijak, “Isoelectric charge of recombinant human follicle-stimulating hormone isoforms determines receptor affinity and in vitro bioactivity,” Endocrinology, vol. 132, pp. 351-356 (1993) discloses the preparation of several isoforms of human recombinant FSH. Each of the isoforms may be considered an FSH analog.

A. Hartee, “Multiple forms of pituitary and placental gonadotropins,” pp. 147-154 in S. Milligan (Ed.), Oxford Reviews of Reproductive Biology (1989) discloses different glycoprotein variants that may be considered analogs of FSH, LH, and CG. Seven isoforms of LH, and six isoforms of hCG were isolated; all had bioactivity in vivo.

Dopamine

M. Samford-Grigsby et al., “Injection of a dopamine antagonist into Holstein steers to relieve symptoms of fescue toxicosis,” J. Anim. Sci., vol. 75, pp. 1026-1031 (1997) describes experiments in which a dopamine antagonist, Ro 24-0409, was observed to reduce fever and to increase serum levels of prolactin in steers suffering from toxicosis after being fed endophyte-infected tall fescue. The first paragraph of the paper references at least four other papers in which various dopamine agonists had previously been used in similar experiments attempting to achieve similar results. See also B. Larson et al., “D₂ dopamine receptor response to endophyte-infected tall fescue and an antagonist in the rat,” J. Anim. Sci., vol. 72, pp. 2905-2910 (1994).

J. Zhang et al., “Effects of dietary protein percentage and 1-agonist administered to prepubertal ewes on mammary gland growth and hormone secretions,” J. Anim. Sci., vol. 73, pp. 2655-2661 (1995) discloses experiments in young ewes using a β-agonist, L-644,969. (Dopamine has both alpha- and beta-adrenergic action. Thus a beta-agonist may be considered a dopamine agonist.)

M. Claeys et al., “Skeletal muscle protein synthesis and growth hormone secretion in young lambs treated with clenbuterol,” J. Anim. Sci., vol. 67, pp. 2245-2254 (1989) discloses experiments in lambs on the effects of clenbuterol, a β-agonist.

Estrogen and Estradiol

N. Adams, “Detection of the effects of phytoestrogens on sheep and cattle,” J. Anim. Sci., vol. 73, pp. 1509-1515 (1995) describes a number of reproductive effects that were attributed to consumption by cattle of forage containing low levels of phytoestrogens, i.e., plant-derived estrogen analogs. Numerous plant sources of various phytoestrogens are described, including isoflavones and coumestans in legumes; various coumestan phytoalexins in infected alfalfa; coumestrol and related compounds in annual medics; various coumestans in infected white clover; various isoflavones in subterranean clover; the isoflavone formononetin in red clover; various isoflavones, as well as coumestrol in soybean. Several specific analogs are described by name, and for some analogs, chemical structures are given as well.

S. Khan et al., “Effects of neonatal administration of diethylstilbestrol in male hamsters: Disruption of reproductive function in adults after apparently normal pubertal development,” Biol. Reprod., vol. 58, pp. 137-142 (1998) discusses the effects of diethylstilbestrol, an estradiol agonist, administered to male hamsters on the day of birth.

J. Richard et al., “Analysis of naturally occurring mycotoxins in feedstuffs and food,” J. Anim. Sci., vol. 71, pp. 2563-2574 (1993) discloses a mycotoxin, zearalenone, that is estrogenic but non-steroidal.

R. Davey et al., “Studies on the use of hormones in lamb feeding I.,” J. Anim. Sci., vol. 18, pp. 64-74 (1940) discloses experiments in lambs involving the use of four estrogenic compounds: stilbestrol, progesterone, benzestrol, and estradiol.

There are a number of naturally-occurring estrogens known in the art (e.g., estrone, estriol, equilin, and equilenin) that would be considered analogs of estradiol. See, e.g., S. Budavari et al. (Eds.), Merck Index, Entries 3581, 3582, 3659, & 3660 (11th Ed. 1989).

W. Isaacson et al., “Testosterone, dihydrotestosterone, trenbolone acetate, and zeranol alter the synthesis of cortisol in bovine adrenocortical cells, J. Anim. Sci., vol. 71, pp. 1771-1777 (1993) discloses in vitro experiments employing testosterone, testosterone analogs and zeranol—the last of which is a synthetic estrogenic compound.

R. Herschler et al., “Production responses to various doses and ratios of estradiol benzoate and trenbolone acetate implants in steers and heifers,” J. Anim. Sci., vol. 73, pp. 2873-2881 (1995) reported experiments in steers and heifers using estradiol benzoate, an estradiol analog, and trenbolone acetate, a testosterone analog.

Somatostatin

Y. Patel et al., “Subtype selectivity of peptide analogs for all five cloned human somatostatin receptors,” Endocrinology, vol. 135, pp. 2814-2817 (1994) reports a study involving 32 different somatostatin analogs. It also reports that two of those somatostatin analogs, SMS 201-995 and BIM 23014, were already in clinical use as long-acting somatostatin preparations as of 1994. References to other papers describing these analogs, as well as commercial sources for specific analogs, were also mentioned.

M. Berelowitz, “Editorial: The somatostatin receptor—a window of therapeutic opportunity?” Endocrinology, vol. 136, pp. 3695-3697 (1995) reported that as of 1995 “a large number of analogs [of somatostatin] with improved stability in plasma” had been synthesized; and also reported that one, octotreotide, was commercially available in the United States, and that two others, lanreotide and somatuline, were in contemporaneous clinical trials.

Melanocyte-Stimulating Hormone

M. Goldman et al., “α-Melanocyte-stimulating hormone-like peptides in the intermediate lobe of the rat pituitary gland: Characterization of content and release in vitro,” Endocrinology, vol. 112, pp. 435-441 (1983) discloses two MSH analogs: desacetyl AMSH; and N, O-diacetyl AMSH.

Testosterone

S. Bartle et al., “Trenbolone acetate/estradiol combinations in feedlot steers: Dose-response and implant carrier effects,” J. Anim. Sci., vol. 70, pp. 1326-1332 (1992) discloses experiments in steers employing trenbolone acetate, a “potent testosterone analog.”

W. Isaacson et al., “Testosterone, dihydrotestosterone, trenbolone acetate, and zeranol alter the synthesis of cortisol in bovine adrenocortical cells, J. Anim. Sci., vol. 71, pp. 1771-1777 (1993) discloses in vitro experiments employing testosterone and the testosterone analogs dihydrotestosterone and trenbolone acetate, as well as zeranol (the last of which is a synthetic estrogenic compound).

R. Herschler et al., “Production responses to various doses and ratios of estradiol benzoate and trenbolone acetate implants in steers and heifers,” J. Anim. Sci., vol. 73, pp. 2873-2881 (1995) reported experiments in steers and heifers using estradiol benzoate, an estradiol analog, and trenbolone acetate, a testosterone analog.

C. Lee et al., “Growth and hormone response of intact and castrate male cattle to trenbolone acetate and estradiol,” J. Anim. Sci., vol. 68, pp. 2682-2689 (1990) reported experiments in steers and intact male cattle using trenbolone acetate, a testosterone analog.

Nanoparticle Cores Useful in the Present Invention

This invention may be practiced with a variety of nanoparticle core materials otherwise known in the art, including silica; alginate; polymers, iron oxides (particularly Fe₃O₄); gadolinium complexes; core-shell nanoparticles such as those disclosed in U.S. patent application Ser. No. 11/054,513, published as United States patent application publication number US-2006-0177660-A1; and quantum dots. The nanoparticle core may take any of the various shapes otherwise known in the art, including for example spheres, rods, prisms, or fibers. The nanoparticle core may optionally include a fluorophore.

Polyanions and Polycations Useful in the Present Invention

This invention may be practiced with a variety of polycations or polyanions. Polycations are used where the embedded compound is anionic, and polyanions are used where the embedded compound is cationic.

Examples of polyanions that may be used in this invention include poly(3,4-ethylenedioxythiophene)-poly(styrenesulfonate) (PEDOT-PSS); poly(vinylpyrrolidone) (PVPON); 2-acrylamido-2-methylpropanesulfonic acid (AMPS); sodium poly(styrenesulfonate) (PSS); protamine (PRM); and bovine serum albumin (BSA).

Examples of polycations that may be used in this invention include poly(allylamine hydrochloride) (PAH); poly(ethyleneimine) (PEI); poly(acrylic acid) (PAA); poly(diallydimethylammonium chloride) (PDADMAC); diazoresin (DR); and dextransulfate (DXS).

Miscellaneous

Nanoparticles in accordance with the present invention may be administered to a patient by any suitable means, including oral, intravenous, parenteral, subcutaneous, intrapulmonary, intranasal administration, or inhalation. The means of administration may depend on the type of cancer or other diseased tissue being targeted. For example, inhalation might be well suited for lung cancers and metastases in the lungs. Intravenous administration will generally be preferred for treating metastases in many other organs, including the brain.

Pharmaceutically acceptable carrier preparations include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, aqueous solutions, emulsions or suspensions, including saline and buffered media. Parenteral vehicles include sodium chloride solution, Ringer's dextrose, dextrose and sodium chloride, lactated Ringer's, or fixed oils. The nanoparticles may be mixed with excipients that are pharmaceutically acceptable and are compatible with the nanoparticles. Suitable excipients include water, saline, dextrose, and glycerol, or combinations thereof. Intravenous vehicles include fluid and nutrient replenishers, electrolyte replenishers, such as those based on Ringer's dextrose, and the like. Preservatives and other additives may also be present such as, for example, antimicrobials anti-oxidants, chelating agents, inert gases, and the like. A preferred carrier is phosphate-buffered saline.

The form may vary depending upon the route of administration. For example, compositions for injection may be provided in the form of an ampoule, each containing a unit dose amount, or in the form of a container containing multiple doses. For clinical use, it is preferred to aliquot the product in lyophilized form, suitable for reconstitution in saline, for preservation and sterility.

Initial in vivo animal trials will be conducted in accordance with all applicable laws and regulations, followed by clinical trials in humans in accordance with all applicable laws and regulations.

Definitions. Unless otherwise clearly indicated by context, the following definitions apply in both the specification and claims.

“Nanoparticle(s)” refer to particle(s) having a mean diameter between about 1 nm and about 1000 nm or between about 5 nm and about 800 nm, preferably between about 100-600 nm or about 20-500 nm. (Note that the “diameter” of a particle refers to its largest dimension, and does not necessarily imply that the particle has a spherical shape or a circular cross section. The particles may, for example, comprise nanofibers, nanorods, nanoprisms, or nanomaterials of other shapes.)

The terms “specific,” “site-specific,” “target-specific,” and “targeted” are interchangeable, and refer to particles that preferentially accumulate in a desired tissue by virtue of compounds on the surface of the particles, for example, compounds such as hormones, ligands, receptors, or antibodies, or fragments thereof that selectively bind to receptors, ligands, or epitopes on the surface of cells in that tissue.

The expression “is essentially free of” is the converse of the term “consists essentially of.” A composition is “essentially free of” a component X either if it contains no X at all, or if small amounts of X are present; but in the latter case, the properties of the composition should be substantially the same (in relevant aspects) as the properties of an otherwise identical composition that is free of X. If sufficient X is present that the properties of the composition are substantially altered (in relevant aspects) as compared to the properties of an otherwise identical composition that is free of X, then the composition is not considered to be “essentially free of” component X.

The term “effective amount” refers to an amount of the specified nanoparticles that is sufficient to selectively kill or inhibit one or more tumors, metastases, nonvascularized malignant cell clusters, or individual malignant cells, or other targeted diseases or cells, to a clinically significant degree; or an amount that is sufficient to deliver an amount of drug to a targeted tissue in a clinically significant amount; in each case without causing clinically unacceptable side effects on non-targeted tissues.

The term “ligand” should be understood to encompass not only the native ligand, but also analogs of the native ligand, including antibodies and antibody fragments against the corresponding receptors. Numerous analogs of many hormones are well known in the art.

Statistical analyses: Unless otherwise indicated, statistical significance is determined by McNemar's test, ANOVA, Student's t-test. Unless otherwise indicated, statistical significance is determined at the P<0.05 level, or by such other measure of statistical significance as is commonly used in the art for a particular type of determination.

Abbreviations: Some of the abbreviations used in the specification:

### LH Luteinizing Hormone

### LHRH Luteinizing Hormone Releasing Hormone

### CG Chorionic Gonadotropin

CG Fragment of the beta chain of CG, amino acid residues 81-95

### FSH Follicle Stimulating Hormone

RES Reticulo-endothelial system

The complete disclosures of all references cited in this specification are hereby incorporated by reference. In the event of an otherwise irreconcilable conflict, the present specification shall control.

What is claimed:

1. A particle comprising an inner core and a outer, multilayer shell; wherein:
(a) at least one dimension of said inner core is between about 1 nm and about 100 nm;
(b) said core and said shell each comprise charged or polar moieties to promote electrostatic binding of said shell to said core;
(c) said multilayer shell comprises a plurality of layers of positively charged compounds and a plurality of layers of negatively charged compounds;
(d) said layers of positively charged compounds and said layers of negatively charged compounds alternate with one another, so that adjacent layers bind to one another electrostatically;
(e) at least one of said charged compounds is a polymer;
(f) at least one of said charged compounds is pharmaceutically active; and
(g) under physiological conditions, the particle will release said pharmaceutically active compound at a half-life between about 1 day and about 20 days.

2. A plurality of particles as recited in claim 1.

3. A particle as recited in claim 1, wherein under physiological conditions, said particle will release said pharmaceutically active compound at a half-life between about 2 days and about 10 days.

4. A particle as recited in claim 1, wherein the free, unbound form of said pharmaceutically active compound aggregates and loses activity under physiological conditions, at a rate substantially faster than the rate at which said pharmaceutically active compound loses activity under physiological conditions when present as a component of said particle.

5. A particle as recited in claim 1, wherein the free, unbound form of said pharmaceutically active compound loses activity under physiological conditions, at a rate substantially faster than the rate at which said pharmaceutically active compound loses activity under physiological conditions when present as a component of said particle.

6. A particle as recited in claim 1, wherein said pharmaceutically active compound has activity against one or more cancers.

7. A particle as recited in claim 1, wherein said pharmaceutically active compound comprises a first domain and a second domain, wherein:
(a) said first domain comprises a hormone selected from the group consisting of gonadotropin-releasing hormone, lamprey III luteinizing hormone releasing hormone (I-LHRH-III), beta chain of luteinizing hormone (βLH), estrogen, testosterone, luteinizing hormone, chorionic gonadotropin, the beta subunit of chorionic gonadotropin, follicle stimulating hormone, melanocyte-stimulating hormone, estradiol, dopamine, somatostatin, and analogues of these hormones; and
(b) said second domain comprises a lytic peptide, wherein said lytic peptide comprises from 10 to 39 amino acid residues, is basic, and will form an amphipathic alpha helix.

8. A particle as recited in claim 1, wherein said pharmaceutically active compound comprises a first domain and a second domain, wherein:
(a) said first domain comprises a hormone selected from the group consisting of corticosteroid-releasing hormone, growth hormone-releasing hormone, vasoactive intestinal polypeptide, pituitary adenylate cyclase activating peptide, MSH, EGF, FSH, Her-2, transferrin, gastrin-releasing peptide, bombesin, Her-2, Her-3, folate, alphaᵥ-beta₃, VEGF, EGF, and analogues of these hormones; and
(b) said second domain comprises a lytic peptide, wherein said lytic peptide comprises from 10 to 39 amino acid residues, is basic, and will form an amphipathic alpha helix.

9. A particle as recited in claim 1, wherein said pharmaceutically active compound comprises Phor21-βCG(ala) (SEQ ID NO 1).

10. A particle as recited in claim 1, wherein said pharmaceutically active compound comprises a lytic peptide or a lytic peptide domain.

11. A particle as recited in claim 1, wherein said particle additionally comprises one or more ligand moieties on the outside of said multilayer shell, wherein said ligand moieties preferentially bind to receptors that are expressed by cells to be selectively targeted by said pharmaceutically active compound.

12. A method comprising administering to a patient a plurality of particles as recited in claim 1, wherein the patient is in need of said pharmaceutically active compound.

13. A method comprising administering to a patient a plurality of particles as recited in claim 3, wherein the patient is in need of said pharmaceutically active compound.

14. A method comprising administering to a patient a plurality of particles as recited in claim 4, wherein the patient is in need of said pharmaceutically active compound.

15. A method comprising administering to a patient a plurality of particles as recited in claim 5, wherein the patient is in need of said pharmaceutically active compound.

16. A method comprising administering to a cancer patient a plurality of particles as recited in claim 6, wherein the patient is in need of said pharmaceutically active compound.

17. A method comprising administering to a cancer patient a plurality of particles as recited in claim 7, wherein the patient has a cancer whose cells express a receptor to which said first domain selectively binds.

18. A method comprising administering to a cancer patient a plurality of particles as recited in claim 8, wherein the patient has a cancer whose cells express a receptor to which said first domain selectively binds.

19. A method comprising administering to a patient a plurality of particles as recited in claim 9, wherein the patient has cancer of the lung, prostate, melanoma, uterine corpus, breast, ovary, testis, or endometrium.

20. A method comprising administering to a patient a plurality of particles as recited in claim 10, wherein the patient is in need of said pharmaceutically active compound.

21. A method comprising administering to a patient a plurality of particles as recited in claim 11, wherein the patient has diseased cells that express a receptor to which said ligand moiety selectively binds.
